(19) United States
(12) Patent Application Publication
(10) Pub. No.: US 2018/0220754 A1
(43) Pub. Date: Aug. 9, 2018

(54) MULTIPLE DEVICE WRIST BAND

(71) Applicant: Matthew STREBE, Cardiff by the Sea, CA (US)

(72) Inventor: Matthew STREBE, Cardiff by the Sea, CA (US)

(21) Appl. No.: 15/579,916

(22) PCT Filed: Jun. 3, 2016
(86) PCT No.: PCT/US16/35860

§ 371 Date: Dec. 5, 2017

Related U.S. Application Data
(60) Provisional application No. 62/171,777, filed on Jun. 5, 2015.

(51) Int. Cl. A44C 5/14; G04G 17/08; G04G 21/02; A61B 5/00

(52) U.S. Cl. CPC A44C 5/14; G04G 17/083; G04G 9/007; A61B 5/681; G04G 21/025

(57) ABSTRACT

A multiple segment wrist band for securing multiple devices to the wrist of a user is provided. The first segment includes a first half of the band clasp on its first end and a first device connector on its second end. The second segment includes a first device connector on its first end and a second device connector on its second end. The third segment includes a second device connector on its first end a second half of the band clasp on its second end. The length of the second segment approximates the curved distance between the top of the user's wrist and the bottom of the user's wrist and thereby securely positions the first device and the second device on opposite sides of the user's wrist when the first and third segments are clasped together.

### BACKGROUND

### Field of the Invention

The present application is generally directed toward a wrist band designed to secure multiple devices to a person's wrist and is more specifically directed toward a dual watch and computing device wristband.

### Related Art

In the field of horology, traditional timepieces function both as functional timekeeping devices and as ornamental jewelry. Haute-horology wrist watches commonly cost more than $10,000 and can range to $1,000,000 new.

Separately, in the field of mobile computing, wearable computing devices often referred to as smart watches are coming into common use. These devices perform timekeeping functions as one of their many features, but are more valued as remote operators of a paired mobile computing device. These smart watches provide alerts such as text message or other service notifications, email viewing, and various other general functions.

However, smart watches are rarely valued for their appearance because they usually present a blank display in order to conserve battery power, and because digital graphical displays are not as desirable as the physical ornamentation of a haute-horology watch.

Currently, users have to choose whether to wear a smart watch or a haute-horology or choose to wear a watch on both wrists, which is not desirable. What is needed is an apparatus that overcomes the significant problems described above.

### SUMMARY

Accordingly, to solve the problems described above the inventor has devised a novel multiple device wrist band that allows a user to simultaneously wear a traditional haute-horology watch and a smart watch. In an embodiment, the novel wrist band may include three separate segments. The first segment can include a first half of the band clasp on its first end and a first device connector on its second end. The second segment can include a first device connector on its first end and a second device connector on its second end. The third segment can include a second device connector on its first end and a second half of the band clasp on its second end. The length of the second segment can approximate the curved distance between the top of the user's wrist and the bottom of the user's wrist and can thereby provide one of a number of ways to securely position the first device and the second device on opposite sides of the user's wrist when the first and third segments are clasped together.

Additional devices can be added to the multiple device wrist band by the inclusion of additional second segment pieces that each include a device connector on both ends.

Other features and advantages of the present invention will become more readily apparent to those of ordinary skill in the art after reviewing the following detailed description and accompanying drawings.

### BRIEF DESCRIPTION OF THE DRAWINGS

The structure and operation of the present invention will be understood from a review of the following detailed description and the accompanying drawings in which like reference numerals refer to like parts and in which:

FIG. 1 is a plan view of an example multiple device wrist band according to an embodiment of the invention;

FIG. 2A is a top view of an example multiple device wrist band according to an embodiment of the invention;

FIG. 2B is a bottom view of an example multiple device wrist band according to an embodiment of the invention; and

FIG. 2C is a side view of an example multiple device wrist band according to an embodiment of the invention.

### DETAILED DESCRIPTION

Certain embodiments disclosed herein provide for a multiple device wrist band. After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention as set forth in the appended claims.

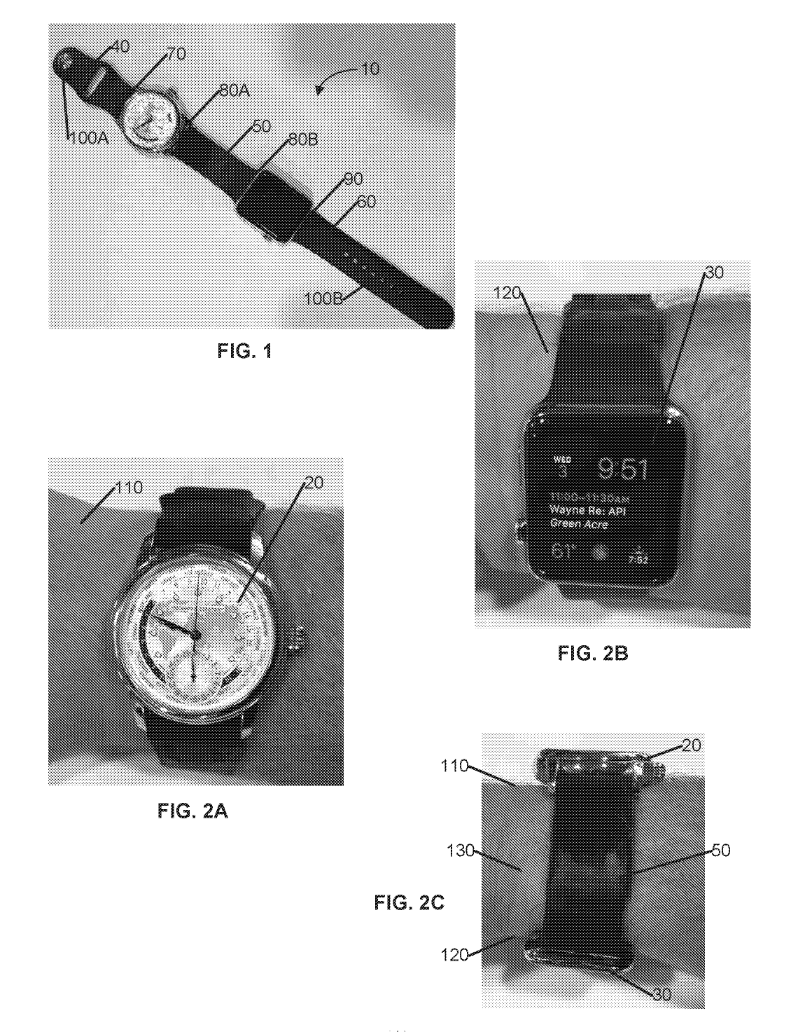

FIG. 1 is a plan view of an example multiple device wrist band 10 according to an embodiment of the invention. In the illustrated embodiment, the band 10 secures a plurality of devices 20 and 30 to the wrist of a user. The band 10 comprises a plurality of segments 40, 50 and 60. Segment 40 has a first portion 100A of a band clasp at its first end and segment 40 is attached to the first device 20 at its second end by way of connector 70. Segment 50 is attached to the first device 20 at its first end by way of connector 80A and is attached to the second device 30 at its second end by way of connector 80B. Segment 60 is attached to the second device 30 at its first end by way of connector 90 and segment 60 has a second portion 100B of the band clasp at its second end.

In operation, the first portion 100A and second portion 100B of the band clasp are joined to secure devices 20 and 30 to the wrist of the user. The length of segment 50 is optimally selected to approximate the curved distance from a portion of the top of the wrist of the user to a portion of the bottom of the wrist of the user. This length advantageously secures the first device 20 on the top of the wrist in the desired location and simultaneously secures the second device 30 on the bottom of the wrist at the desired location. In one embodiment, the location of the second device 30 on the bottom of the wrist improves the second device's interrogation of user biometrics by orienting the second device 30 closer to the blood vessels and arteries within the wrist and by reducing or eliminating interference caused by internal structures such as the wrist and forearm bones.

In an alternative embodiment, the multiple-device wrist band 10 comprises a single band segment (i.e., 40/50/60 combined) to which multiple devices are secured. In another alternative embodiment, the multiple-device wrist band 10 comprises two band segments (i.e., 40 and 50/60 combined OR 40/50 combined and 60) to which multiple devices are secured.

FIG. 2A is a top view of an example multiple device wrist band 10 according to an embodiment of the invention. In the illustrated embodiment, the device 20 is secured to the top 110 of the user's wrist.

FIG. 2B is a bottom view of an example multiple device wrist band 10 according to an embodiment of the invention. In the illustrated embodiment, the device 30 is secured to the bottom 120 of the user's wrist.

FIG. 2C is a side view of an example multiple device wrist band 10 according to an embodiment of the invention. In the illustrated embodiment, the device 20 is secured to the top 110 of the user's wrist and simultaneously the device 30 is secured to the bottom 120 of the user's wrist. The multiple devices 20 and 30 are positioned on the top 110 and bottom 120 of the user's wrist by band segment 50.

### ADVANTAGES

An advantage of the present application is the ability to use all of the features of both a haute-horology watch and a smart watch. For example, smart watches, which conserve power by turning off the display, must be awakened by the user in order to observe the time, either by touching the display or moving the watch in a specific motion. This makes it difficult for the wearer to discreetly observe the time. Conspicuously observing the time is frequently interpreted by others as boredom or a desire to leave, and smart watch wearers may be dissatisfied by the inability of a smart watch to display the time consistently without requiring movement or activating the display and using precious battery power. Accordingly, wearing a traditional timepiece along with a smart watch is desirable.

Another advantage of the present application is that the smart watch can be worn on the inside of the wrist, thus protecting notifications from open disclosure and keeping them discreetly for the owner rather than presented publically on the top of the wrist.

Another advantage of the present application is that the smart watch need not attempt to mimic haute-horology watches by being unreasonably expensive or using exotic materials for a device whose service life and time to obsolescence is much shorter than a haute-horology watch.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

1. A band apparatus that secures multiple devices to a wrist of a user, the band apparatus comprising a single band to which the multiple devices are affixed.

2.-6. (canceled)

7. An apparatus comprising:
a first band segment having a first end and a second end, the first end of the first band segment comprising a first portion of a band clasp and the second end of the first band segment comprising a first device connector configured to releasably attach to a first wearable device at a first area of the first device;
a second band segment having a first end and a second end, the first end of the second band segment comprising a first device connector configured to releasably attach to the first wearable device at a second area of the first device and the second end of the second band segment comprising a second device connector configured to releasably attach to a second wearable device at a first area of the second device;
a third band segment having a first end and a second end, the first end of the third band segment comprising a second device connector configured to releasably attach to the second wearable device at a second area of the second device and the second end of the third band segment comprising a second portion of the band clasp, wherein
the first portion of the band clasp and the second portion of the band clasp are configured to attach to each other to secure the apparatus to a wrist of a user.

8. The apparatus according to claim 7, wherein at least one of the multiple devices is a timepiece.

9. The apparatus according to claim 7, wherein at least one of the multiple devices is a multifunction computing device.

10. The apparatus according to claim 7, wherein a first of the multiple devices is positioned on an upper side of the wrist of the user and a second of the multiple devices is positioned on a lower side of the wrist of the user.

11. The apparatus according to any of claims 7, wherein the first wearable device is a traditional timepiece.

12. The apparatus according to any of claims 11, wherein the second wearable device is a smart watch.

13. The apparatus according to any of claims 7, wherein the length of the second band segment approximates the curved distance between the top of the user's wrist and the bottom of the user's wrist and thereby securely positions the first wearable device and the second wearable device on opposite sides of the user's wrist when the first and third band segments are clasped together.

14. The apparatus according to any of claims 13, wherein the position of the second wearable device on the bottom of the user's wrist improves the second wearable device's interrogation of user biometrics.

15. The apparatus according to any of claims 14, wherein the second wearable device's interrogation of user biometrics are improved by orienting the second wearable device closer to the blood vessels and arteries within the wrist of the user.

16. The apparatus according to any of claims 15, wherein the second wearable device's interrogation of user biometrics are improved by reducing or eliminating interference caused by internal structures such as the wrist bones and forearm bones.

17. An apparatus comprising:
a first band segment having a first end and a second end, the first end of the first band segment comprising a first portion of a band clasp and the second end of the first band segment comprising a second portion of the band clasp;
wherein a first portion of the first band segment is configured to extend through one or more first device connectors positioned at one or more areas of a first wearable device to secure the first wearable device to the first band segment, and
wherein a second portion of the first band segment is configured to extend through one or more second device connectors positioned at one or more areas of a second wearable device to secure the second wearable device to the first band segment, wherein
the first portion of the band clasp and the second portion of the band clasp are configured to attach to each other to secure the apparatus to a wrist of a user.

18. The apparatus according to claim 17, wherein at least one of the multiple devices is a timepiece.

19. The apparatus according to claim 17, wherein at least one of the multiple devices is a multifunction computing device.

20. The apparatus according to claim 17, wherein a first of the multiple devices is positioned on an upper side of the wrist of the user and a second of the multiple devices is positioned on a lower side of the wrist of the user.

21. An apparatus comprising:
a first band segment having a first end and a second end, the first end of the first band segment comprising a first portion of a band clasp and the second end of the first band segment comprising a first device connector configured to releasably attach to a first wearable device at a first area of the first device;
a second band segment having a first end and a second end, the first end of the second band segment comprising a first device connector configured to releasably attach to the first wearable device at a second area of the first device and the second end of the second band segment comprising a second portion of the band clasp,
wherein the second band segment is configured to extend through one or more second device connectors positioned at one or more areas of a second wearable device to secure the second wearable device to the apparatus, and
wherein the first portion of the band clasp and the second portion of the band clasp are configured to attach to each other to secure the apparatus to a wrist of a user.

22. The apparatus according to claim 21, wherein at least one of the multiple devices is a timepiece.

23. The apparatus according to claim 21, wherein at least one of the multiple devices is a multifunction computing device.

24. The apparatus according to claim 21, wherein a first of the multiple devices is positioned on an upper side of the wrist of the user and a second of the multiple devices is positioned on a lower side of the wrist of the user.
